(19) United States
(12) Patent Application Publication
(10) Pub. No.: US 2006/0009515 A1
(43) Pub. Date: Jan. 12, 2006

(54) Process and intermediates for preparing escitalopram

(75) Inventor: Hoi Lun Allan Tse, Ontario (CA)

Correspondence Address: SHLESINGER, ARKWRIGHT & GARVEY LLP, 1420 KING STREET, SUITE 600, ALEXANDRIA, VA, 22314, US

(73) Assignee: TORCAN CHEMICAL LTD, ONTARIO (CA)

(21) Appl. No.: 10/510,890

(22) PCT Filed: Apr. 8, 2003
(86) PCT No.: PCT/CA03/00522

§ 371 Date: Mar. 11, 2005

(30) Foreign Application Priority Data
Apr. 9, 2002 (CA) 2381341

(51) Int. Cl. C07D 307/78; A61K 31/343

(52) U.S. Cl. 514/469; 549/467

(57) ABSTRACT

The antidepressant drug Escitalopram is prepared from 5-bromophthalide via the diol intermediate (4-bromo-2-(hydrox-ymethyl)phenyl)-(4-fluorophenyl)methanol. The racemic diol intermediate is converted to an enantiomerically enriched form by first converting the diol to a monoester intermediate and then reacting the monoester intermediate with an optically active acid, most preferably (+)-di-p-toluoyl tartaric acid, to form a salt. The salt is then crystallized to recover an enantiomerically enriched, crystalline form thereof. The monoester intermediate is preferably formed by reacting the racemic diol intermediate with an acid or a reactive acid derivative which, in a particularly preferred embodiment, is acetic anhydride.

### FIELD OF THE INVENTION

The invention relates to processes and intermediates for preparing the antidepressant drug Escitalopram.

### BACKGROUND OF THE INVENTION

Escitalopram is the S-(+) enantiomer of 1-(3-dimethylaminopropyl)-1-(4-fluorophenyl)-1,3-dihydroisobenzofuran-5-carbonitrile, and is known to be useful as an antidepressant. Racemic 1-(3-dimethylaminopropyl)-1-(4-fluorophenyl)-1,3-dihydroisobenzofuran-5-carbonitrile is also known as Citalopram.

Two synthetic routes for preparing Citalopram are disclosed in U.S. Pat. No. 3,467,675 (Petersen et al.), issued on Sep. 16, 1969. A first process, disclosed at column 2, lines 5 to 26, comprises dehydration of a 1-dialkylaminoalkyl diol intermediate (general formula II of the Petersen et al. patent). This intermediate is prepared by subjecting a halo-phthalide compound (general formula II) to two consecutive Grignard reactions as described at column 3, lines 9 to 52. A second process, described at column 4, lines 1 to 39, involves dehydration of a diol intermediate, followed by reaction of the cyclized product with a dialkylaminoalkyl halide.

Canadian Patent No.1,237,147 (Boegesoe), issued on May 24, 1988, describes a process for preparing Citalopram from 5-cyanophthalide via a 5-cyano -1-dimethylaminopropyl diol intermediate analogous to general formula II of the above-mentioned Petersen et al. patent. Canadian Patent Nos. 1,339,452 (Boegesoe), issued on Sep. 9, 1997 and 1,339,468 (Boegesoe et al.), issued on Dec. 2,1997, disclose resolution of the 5-cyano-1-dimethylaminopropyl diol intermediate into its enantiomers by converting the diol into a monoester of an optically active carboxylic acid, and separating the enantiomers by HPLC or fractional crystallization. The S-(+)enantiomer of the diol intermediate is then converted to Escitalopram by ring closure with conservation of stereoconfiguration.

The need exists for alternate synthetic routes for preparation of Escitalopram.

### SUMMARY OF THE INVENTION

The present invention provides an alternative synthesis of Escitalopram which begins with 5-bromophthalide and proceeds via the diol intermediate (4-bromo-2-(hydroxymethyl)phenyl)-(4-fluorophenyl)methanol. According to the process of the invention, the racemic diol intermediate is converted to an enantiomerically enriched form by first converting the diol to a monoester, intermediate and then reacting the monoester intermediate with an optically active acid to form a salt. The salt is then crystallized to recover an enantiomerically enriched, crystalline form thereof.

Preferably, the monoester intermediate is formed by reacting the racemic diol intermediate with an acid or a reactive acid derivative, the acid or derivative thereof being selected to yield a crystalline salt. In a particularly preferred embodiment, the racemic diol intermediate reacts with acetic anhydride, which yields the highly crystalline monoacetate ester of the diol intermediate, a novel compound.

Similarly, the optically active acid is selected to yield a salt which is highly crystalline and which is enriched in an enantiomer thereof. In a particularly preferred embodiment of the invention, the optically active acid is (+)-di-p-toluoyl tartaric acid.

After isolation in an enantiomerically enriched, crystalline form, the salt is neutralized and hydrolyzed to yield the optically active diol intermediate, which is then converted to Escitalopram by dehydration and by replacement of the 5-bromo group by a nitrile function.

In one aspect, the present invention provides a process for preparing escitalopram, comprising: (a) reacting 5-bromophthalide with 4-fluoro-phenylmagnesium bromide to produce 4-bromo-2-hydroxymethyl-4′-fluorobenzophenone; (b) reacting said 4-bromo-2-hydroxymethyl4′-fluorobenzophenone with 3-dimethylaminopropyl magnesium chloride to produce racemic (4-bromo-2-(hydroxymethyl)phenyl)-(4-fluorophenyl)methanol; (c) converting said racemic (4-bromo-2-(hydroxymethyl)phenyl)-(4-fluorophenyl)methanol to a racemic monoester intermediate by reaction with a carboxylic acid or a reactive derivative thereof; (d) reacting said racemic monoester intermediate with an optically active acid to form a salt of said racemic monoester intermediate; (e) crystallization of said salt to recover an enantiomerically enriched, crystalline form of said salt; (f) neutralization of said salt to give an enantiomerically enriched form of said monoester intermediate; (g) hydrolysis of the enantiomerically enriched form of said monoester intermediate to produce enantiomerically enriched (4-bromo-2-(hydroxymethyl)phenyl)-(4-fluorophenyl)methanol; (h) dehydration of said enantiomerically enriched (4-bromo-2-(hydroxymethyl)phenyl)-(4-fluorophenyl)methanol to produce enantiomerically enriched 1-(4′-fluorophenyl)-1-(3-dimethylaminopropyl)-5-bromophthalane; and (i) replacement of bromine by a nitrile group to produce escitalopram.

In another aspect, the present invention provides a process for preparing enantiomerically enriched (4-bromo-2-(hydroxymethyl)phenyl)-(4-fluorophenyl)methanol from racemic (4-bromo-2-(hydroxymethyl)phenyl)-(4-fluorophenyl)methanol, comprising: steps (c) to (g) above.

### BRIEF DESCRIPTION OF THE DRAWINGS

The invention will now be described, by way of example only, with reference to the accompanying drawings in which:

FIG. 1 is a reaction scheme showing a preferred synthetic route according to the present invention for preparing Escitalopram;

FIG. 2 is a reaction scheme showing the steps involved in separating the diol intermediate, (4-bromo-2-(hydroxymethyl)phenyl)-(4-fluorophenyl)methanol, into its enantiomers; and

FIG. 3 shows a reaction scheme for preparing the 5-bromophthalide starting material.

### DETAILED DESCRIPTION OF PREFERRED EMBODIMENTS

A preferred process for preparing Escitalopram will now be described below with reference to the attached drawings.

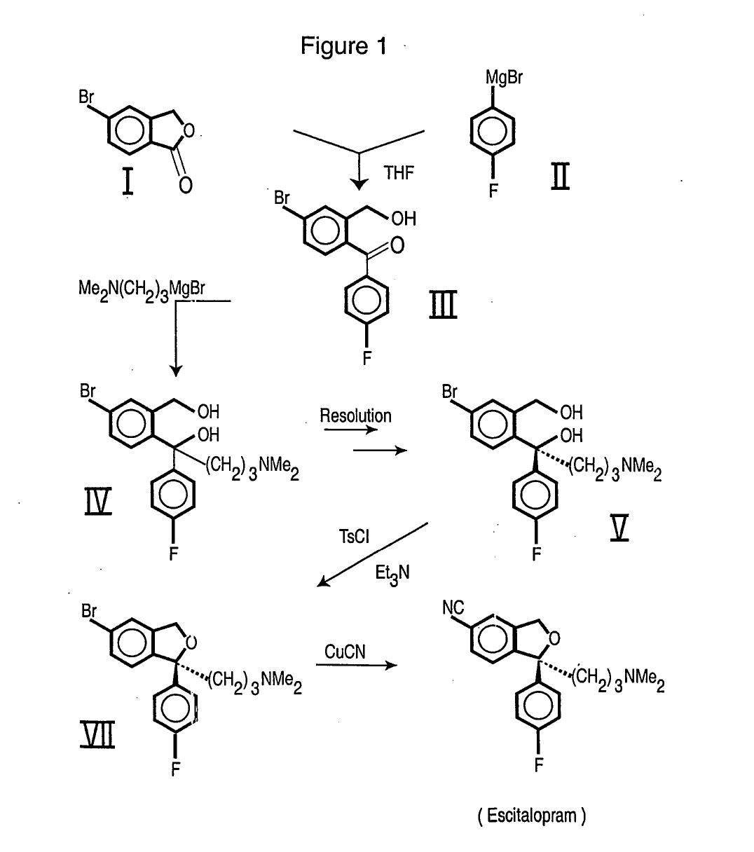

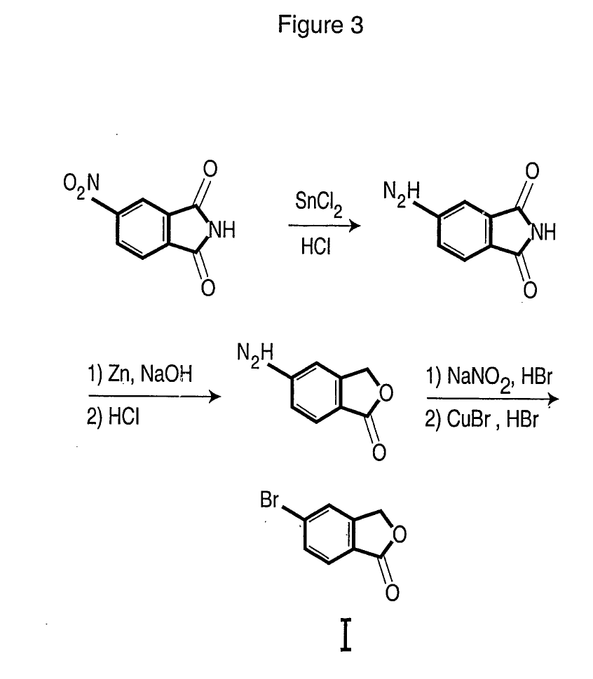

As illustrated in FIG. 1, the starting material in the process for preparing Escitalopram according to the invention is 5-bromophthalide, identified as Formula I in FIG. 1. The 5-bromophthalide used in the synthesis can either be obtained commercially or can be prepared by known synthetic routes. One synthesis of 5-bromophthalide is described in J. Chem. Soc. (1931) pp. 79, 867-870, and is illustrated in FIG. 3. This synthesis comprises three steps, starting with 4-nitrophthalimide, the overall yield typically being about 40 percent.

The first step in the synthesis of Escitalopram comprises reaction of 5-bromophthalide with the Grignard reagent 4-fluorophenyl magnesium bromide, identified as formula II in FIG. 1. The Grignard reagent is prepared in a conventional fashion by reacting 4-fluoro-bromobenzene with magnesium. The product of this reaction is 4-bromo-2-hydroxymethyl4′-fluorobenzophenone, identified as formula III in FIG. 1. Preferably, this compound is not isolated prior to further reaction. This first step in the synthesis of Escitalopram is disclosed in above-mentioned U.S. Pat. No. 3,467,675 and in Canadian Patent No. 1,094,087 (Boegesoe et al.) which issued on Jan. 20, 1981, both of which are incorporated herein by reference.

The second step in the process shown in FIG. 1 is the reaction of the 4-bromo-2-hydroxymethyl-4′-fluorobenzophenone produced in the first step with the Grignard reagent 3-dimethylaminopropyl magnesium chloride to give racemic (4-bromo-2-(hydroxymethyl)phenyl)-(4-fluorophenyl)methanol, identified as formula IV in FIG. 1 and also referred to herein as the “racemic diol intermediate”. This step is also disclosed in U.S. Pat. No. 3,467,675 and Canadian Patent No. 1,094,087.

The 3-dimethylaminopropyl magnesium chloride is prepared in a conventional manner by reaction of 3-dimethylaminopropyl chloride with magnesium.

The inventors have found that the above-described first and second steps of the synthesis are best carried out in tandem without isolation of the reaction product of the first step, namely 4-bromo-2-hydroxymethyl-4′-fluorobenzophenone. Preferably, the 5-bromophthalide is first reacted with 4-fluorophenyl magnesium bromide in THF at a temperature at or below room temperature. After the reaction is complete, the 3-dimethylaminopropyl magnesium chloride is added to the reaction mixture and the resulting mixture is preferably refluxed until completion of the reaction.

The racemic (4-bromo-2-(hydroxymethyl)phenyl)-(4-fluorophenyl)methanol produced by the first two steps is preferably isolated from the reaction mixture, for example by column chromatography. The product is an oil and is typically obtained in a yield of about 50 percent, calculated from the 5-bromophthalide starting material.

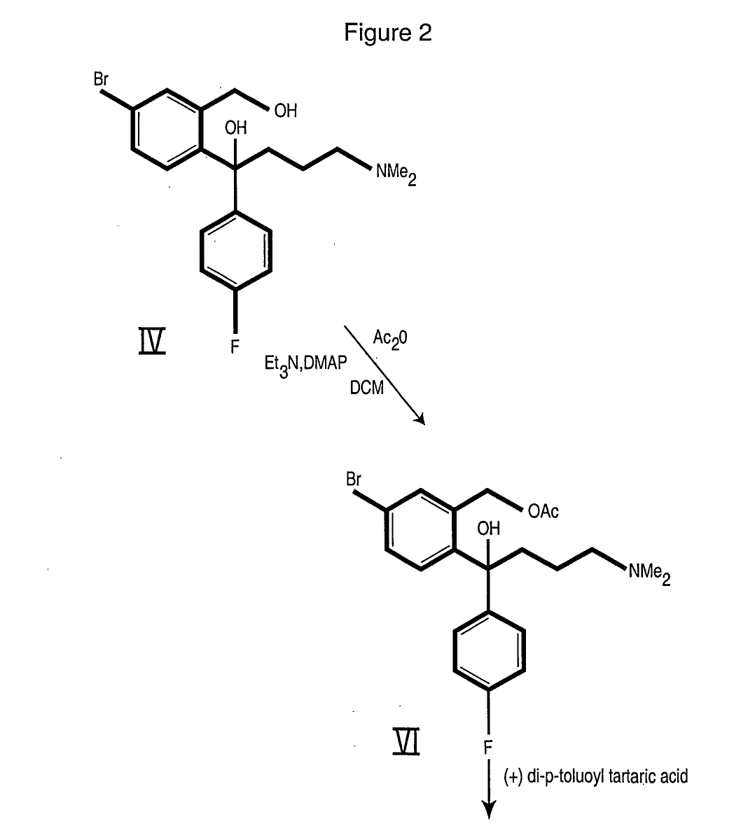

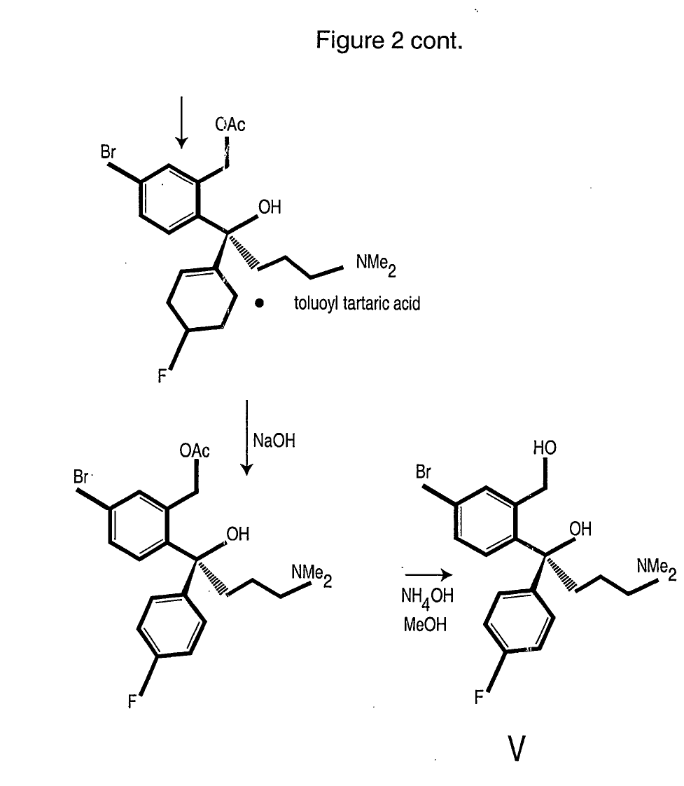

As shown in FIG. 1, the next step in the process comprises resolution of racemic (4-bromo-2-(hydroxymethyl)phenyl)-(4-fluorophenyl)methanol to obtain the (+)-enantiomer of (4-bromo-2-(hydroxymethyl)phenyl)-(4-fluorophenyl)methanol in substantially pure form. This compound is identified as formula V in FIG. 1, and is also referred to herein as the “optically active diol intermediate”. The preferred steps followed during resolution of the racemic (4-bromo-2-(hydroxymethyl)phenyl)-(4-fluorophenyl)methanol are now discussed below with reference to FIG. 2.

The first step in the resolution of racemic (4-bromo-2-(hydroxymethyl)phenyl)-(4-fluorophenyl)methanol involves conversion of racemic (4-bromo-2-(hydroxymethyl)phenyl)-(4-fluorophenyl)methanol to a racemic ester intermediate by reaction with a carboxylic acid or a reactive derivative thereof, such as a halide or anhydride of a carboxylic acid. Preferably the carboxylic acid or reactive derivative thereof is selected to generate a crystalline ester derivative of racemic (4-bromo-2-(hydroxymethyl)phenyl)-(4-fluorophenyl)methanol

In a particularly preferred embodiment of the invention illustrated in FIG. 2, racemic (4-bromo-2-(hydroxymethyl)phenyl)-(4-fluorophenyl)methanol is reacted with acetic anhydride to form the racemic monoacetate ester intermediate identified in FIG. 2 as formula VI. The inventors have found the racemic monoacetate ester intermediate to be a highly crystalline material which is preferably isolated by crystallization from the reaction mixture. The yield of the isolated intermediate is typically about 60 percent.

The inventors have found that conversion of the racemic monoacetate ester intermediate to a crystalline diastereomeric salt by reaction with an optically active acid, followed by isolation of the salt, can lead to production of the (+)-enantiomer of (4-bromo-2-(hydroxymethyl)phenyl)-(4-fluorophenyl)methanol of high optical purity. According to a particularly preferred method of the invention, the racemic monoacetate ester intermediate is reacted with di-p-toluoyl tartaric acid, most preferably with (+)-di-p-toluoyl tartaric acid, to produce a salt which can be isolated as a crystalline solid and purified by recrystallization to yield the diastereomeric salt in highly pure form, typically greater than 95 percent optical purity.

The crude yield of the diastereomeric salt is typically about 15 percent, with the optical purity of the crude salt typically being from about 85 to 90 percent. The purity of the crude salt is subsequently increased by recrystallization, preferably from acetone/hexanes or ethyl acetate, more preferably from acetone/hexanes, with the optical purity of the recrystallized salt typically being greater than 95 percent.

The purified salt is then converted to the optically active diol intermediate by neutralization of the salt and hydrolysis of the acetate monoester function. The neutralization is preferably performed by addition of base, for example dilute sodium hydroxide, and the ester is preferably hydrolyzed with ammonium hydroxide.

Returning to FIG. 1, the optically active diol intermediate of formula V is dehydrated to effect ring closure, thereby producing optically active 1-(4′-fluorophenyl)-1-(3-dimethylaminopropyl)-5-bromophthalane identified by formula VII in FIG. 1. The dehydration may be carried out by any of the procedures described in Canadian Patent No. 1,094,087 and U.S. Pat. No. 3,467,675. However, the inventors prefer carrying out the dehydration with p-toluenesulfonyl chloride, also known as tosyl chloride, and referred to in FIG. 1 as “TsCl”. The cyclized product is typically obtained in a yield of about 80 percent.

The final step of the process comprises replacement of the bromine in 1-(4′-fluorophenyl)-1-(3-dimethylaminopropyl)-5-bromophthalane by a nitrile group to yield Escitalopram which is identified as formula VIII in FIG. 1. The replacement of bromine by the nitrile group is preferably achieved by reaction with cuprous cyanide (CuCN) in the manner disclosed by Canadian Patent No. 1,094,087, with the yield of Escitalopram typically being about 60 percent.

The invention is further illustrated by the following examples:

### EXAMPLE 1

### Preparation of Racemic(4-bromo-2-(hydroxymethyl)phenyl)-(4-fluorophenyl)methanol

To a stirred suspension of 5-bromophthalide (21.3 g, 0.1 mol) in dry THF (120 mL) under an atmosphere of nitrogen at −20° C. was added slowly a 1 M solution of 4-fluorophenylmagnesium bromide in THF (110 mL, 1.1 eq). The reaction temperature of the reaction mixture during addition was kept below −15° C. A thick dark green solution was formed which was stirred moderately for 3 hours while the temperature of the reaction mixture was allowed to warm to room temperature. The mixture was stirred for another 22.5 hours at room temperature. A creamy green coloured suspension was obtained.

To the above suspension was added a 1.48 M solution of 3-dimethylaminopropyl magnesium chloride in THF (see Example 2 for its preparation) (76 mL, 1.12 eq) to generate a thick dark green solution. This solution was refluxed under nitrogen with moderate stirring for 4 hours. The reaction mixture was cooled to room temperature and then was immersed in an ice-water bath. Saturated NH₄Cl solution (100 mL) was added into the mixture in one batch. The resulting mixture was stirred vigorously for 5 minutes. The cooling bath was removed and the mixture was diluted with water (50 mL) and ethyl acetate (EtOAc) (50 mL). To aid phase separation, hexanes (50 mL) and brine (50 mL) were also added. The resulting mixture was transferred to a separatory funnel and rinsed forward with 50 mL of EtOAc. The lower aqueous phase was removed, saturated with NaCl and then was extracted with EtOAc (100 mL). All organic phases were combined and dried over anhydrous Na₂SO₄. Removal of the solvent under reduced pressure gave the crude product (˜38 g) as a thick syrup.

The crude product was purified by passing it through a short column of silica gel 60 (400 g, 230-400 mesh size) packed with EtOAc. The column was eluted, sequentially, with 1.35 L of EtOAc, 2 L of 10% (10% NH₄OH in MeOH) in EtOAc, and 1 L of 20% (10% NH₄OH in MeOH) in EtOAc. Fractions of about 250 mL were collected. Appropriate fractions were combined and the solvent was evaporated under reduced pressure to give a brown oil. Drying this oil under vacuum afforded 20.7 g (52%) of the racemic diol intermediate as a light brown syrup.

### EXAMPLE 2

### Preparation of 3-dimethylaminopropylmagnesium chloride

To a 500 mL round-bottomed flask (“rbf”) equipped with a stirring bar was charged the commercially available 3-dimethylaminopropyl chloride hydrochloride (50 g, 0.315 mol) as a white solid. An aqueous 3 M solution of NaOH (120 mL, ˜1.1 eq) was added to the solid followed by stirring moderately for 15 minutes to generate an oily suspension. Methyl-t-butyl ether (MTBE) (100 mL) was added to this suspension followed by stirring for 10 minutes. The top MTBE layer was collected. The lower aqueous layer was extracted with 50 mL of MTBE. The combined MTBE layers were washed with water (20 mL). This MTBE solution of the amine was dried under azeotropic conditions under nitrogen for 4 hours to remove residual water. MTBE (125 mL) was then distilled out. The contents of the pot were transferred to a 100 mL rbf and the weight of this solution was determined. This solution was then subjected to distillation at atmospheric pressure (bath temperature 80°-90° C.) to remove the remaining MTBE. The content remained was then distilled at the same bath temperature but under reduced pressure (aspirator). The distillate (31.86 g) was then assayed by ¹H nmr to determine the amount of residual MTBE present with respect to 3-dimethylaminopropyl chloride. It was found that the distillate contains about 3% by weight of MTBE.

A 250 mL flame-dried 3-necked rbf equipped with a magnetic stirring bar, a reflux condenser, and a rubber septum was charged with magnesium turnings (2.94 g). The setup was flushed with nitrogen. Sufficient THF (15 mL) was added to cover the magnesium. About 2 mL of 3-dimethylaminopropyl chloride (containing about 3% by weight of MTBE) was also added, together with 3 drops (from a 20 gauge needle) of 1,2-dibromoethane and a few crystals of iodine. The Grignard reaction was initiated with heating using a heat gun and the reaction mixture started to reflux. Once the reaction was initiated (iodine colour disappeared), 3-dimethylaminopropyl chloride and THF were added concurrently to the reaction mixture at a rate such that a gentle reflux was maintained without external heating. The total amount of 3-dimethylaminopropyl chloride and THF used for this reaction are 14.06 g and 65 mL, respectively. After the addition of the reagent and solvent were completed, the reaction mixture was refluxed for 30 minutes. The solution was cooled to room temperature under nitrogen. A 0.15 mL aliquot of the solution was withdrawn and quenched into 0.75 g of CD₃OD in a flame-dried 10 mL rbf under nitrogen. Using ¹H nmr spectroscopy, the amount of the Grignard reagent present in the mixture was determined by analyzing the content in the CD₃OD. The total volume of the Grignard reagent in THF was measured with a syringe (76 mL) and the strength of the reagent was calculated. In this preparation, the concentration of the Grignard reagent solution was found to be about 1.48 M. This solution was used for reaction with 4-bromo-2-hydroxymethyl4′-fluorobenzophenone in situ.

### EXAMPLE 3

### Preparation of monoacetate ester of the racemic diol intermediate

To a cold (0° C.) stirred solution of the racemic diol intermediate of Example 1 (20.7 g, 0.0523 mol) in CH₂Cl₂ (100 mL) containing Et₃N (8.74 mL, 1.2 eq) and 4-dimethylaminopyridine (DMAP) (319 mg, 0.05 eq) was added slowly acetic anhydride (Ac₂O) (5.43 mL, 1.1 eq) over a period of 5 minutes under nitrogen. After the addition, the cooling bath was removed and stirring of the resultant mixture was continued for 67 hours (over the weekend). The reaction was quenched by the addition of 2 mL of MeOH followed by stirring for 1 hour. The mixture was concentrated on a rotary evaporator to give a thick brown oil, which was taken up in 150 mL of EtOAc to produce a brown solution. This solution was washed with 50 ml of 50% conc. NH₄OH in a separatory funnel. An emulsion was formed. This emulsion was broken up by the addition of 50 mL of water and 30 mL of hexanes. The aqueous layer was removed and the organic layer was washed twice with 50 mL of saturated NH₄Cl solution, once with 50 mL of brine and then was dried over anhydrous MgSO₄ and Na₂SO₄. Phase separation occurred. The supernatant was transferred back to a separatory funnel and the lower aqueous layer was discarded. The upper organic layer was again dried over anhydrous MgSO₄ and Na₂SO₄. Removal of the drying agent followed by evaporation of the solvent provided a thick brown oil, which solidified on standing. This solid was redissolved in EtOAc and then concentrated on a rotary evaporator to give a brown oil again. This oil was stirred gently with a stirring bar while MTBE was added gradually into the oil. A total of 30 mL of MTBE was added. White solid started to crystallize out of the solution resulting in the formation of a heavy suspension, which was stirred for 30 minutes. The crystalline solid formed was collected by suction filtration and was dried under vacuum to give 13.27 g (58%) of a beige coloured powder. The filtrate was concentrated to give a brown oil, which solidified on standing. This material was not processed further.

### EXAMPLE 4

### Preparation of the (+)-di-p-toluoyl tartaric acid salt of the monoacetate ester of the diol Intermediate

To a 500 mL rbf was charged the crystallized monoacetate ester of the racemic diol intermediate produced in Example 3 (13.52 g, 0.0309 mol) and (+)-di-p-toluoyl tartaric acid (11.92 g, 1 eq). Acetone (135 mL) was added to the solid to give a heavy white suspension. With moderate stirring, the mixture was brought to reflux under nitrogen. All the solid dissolved and a pale brown solution was obtained. Heating was stopped and the solution was allowed to cool to room temperature. The acetone was removed under reduced pressure to produce a beige foam, which was dissolved in 100 mL of EtOAc to give a brown solution. This solution was stirred gently at room temperature and crystalline white solid started to form quickly. After stirring for 20 minutes, a heavy white suspension was obtained. Another 50 mL of EtOAc was added to aid stirring. This suspension was heated to reflux under nitrogen and EtOAc was added in small portions to dissolve the solid. After 44 mL of EtOAc was added, a clear brown solution was obtained. The total amount of EtOAc used was 194 mL. Heating was stopped and the solution was allowed to cool slowly to room temperature with gentle stirring. After 40 minutes at room temperature, 10 mL of hexane was added to induce crystallization and the solution was left standing over the weekend (total time ˜65 hours), during which time a white suspension formed. The solid precipitate was collected by suction filtration and dried under vacuum to give 3.71 g (14.6% recovery) of the enriched salt as a white powder. ¹H nmr analysis of this solid indicated that it was a 12:88 mixture of the two diastereomeric salts. The filtrate was concentrated to give a brown foam, which was not processed further.

### EXAMPLE 5

### Recrystallization of the (+)-di-p-toluoyl tartaric acid salt of the monoacetate ester of the diol Intermediate

To a 500 mL rbf was charged 4.36 g of the crude diastereomeric salt produced in Example 4 (13:87 diastereomer ratio) followed by a 2:1 mixture of acetone-hexane (100 mL). With gentle stirring, this suspension was brought to reflux under nitrogen. More solvent (2:1 mixture of acetone-hexane) was added gradually to the suspension at reflux until a clear solution was obtained. Total amount of solvent used was 230 mL (100 mL+130 mL). Hexane was then added to this solution at reflux in four 19 mL portions to generate a heavy white suspension. The final ratio of acetone to hexane in the solvent became 1:1. Heating was stopped. The heavy suspension obtained was allowed to cool to room temperature slowly in the oil bath with gentle stirring over a period of 1 hour followed by stirring at room temperature for another 1.5 hours. The white solid was collected by suction filtration and was dried under vacuum to give 2.84 g (65% recovery) of a white powder. ¹H nmr analysis of this solid showed an isomer ratio of 3:97. The filtrate was concentrated to give a pale yellow foam, which was not processed further.

### EXAMPLE 6

### Preparation of the Optically Active Diol Intermediate

The highly enriched salt (2.84 g, 3.436 mmol) obtained in Example 5 was suspended in 40 mL of EtOAc. With moderate stirring, 20 mL of a 0.5 M solution of NaOH (3 eq) was added. The resulting mixture was stirred for 15 minutes at room temperature. This biphasic mixture was transferred to a separatory funnel and the aqueous layer was removed. The organic layer was washed with 10 mL of brine and then was dried over anhydrous Na₂SO₄. Removal of the drying agent followed by evaporation of solvent gave 1.45 g of the optically active monoacetate of the diol intermediate as a thick oil.

This compound (1.45 g) was then dissolved in 9 mL of MeOH. To this solution was added 1 mL of conc. NH₄OH. The resulting solution was stirred for 10 minutes at which time a heavy white precipitate was formed. After a total time of 1.5 hours at room temperature, more MeOH (6 mL) and conc. NH₄OH (2 mL) were added to dissolve the solid precipitated. Stirring was continued for another 2 hours and 20 minutes. Analysis of an aliquot of the reaction mixture indicated that about 10% of the acetate remained.

After a total reaction time of 6 hours, another 1 mL of conc. NH₄OH was added and stirring was continued for another 0.75 hour (total reaction time=6.75 hours). The solvent was removed on a rotary evaporator and the residue formed was dissolved in 20 mL of EtOAc. This solution was washed with brine and then was dried over anhydrous Na₂SO₄. Removal of the drying agent followed by evaporation of solvent gave crude optically active diol which was dried under vacuum to provide 1.356 g (quantitative) of the optically active diol intermediate as a thick oil.

### EXAMPLE 7

### Preparation of 1-(4′-fluorophenyl)1-(3-dimethylaminopropyl)-5-bromophthalane

To a stirred solution of the diol intermediate (1.356 g, 3.424 mmol) produced in Example 6 in 10 mL of CH₂Cl₂ at room temperature was added Et₃N (0.573 mL, 1.2 eq) and p-toluenesulfonyl chloride (718 mg, 1.1 eq). The resulting solution was stirred for 19.5 hours. The reaction was stopped by the addition of 0.5 mL of MeOH followed by stirring for 1 hour. The solvent was removed to give a white solid residue, which was dissolved in EtOAc (30 mL). The resultant solution was stirred with 15 mL of a 0.5 M solution of NaOH for 10 minutes. With the aid of a separatory funnel, the aqueous phase was removed and the organic layer was washed twice with 10 mL portions of saturated NH₄Cl solution, resulting in formation of an emulsion. The organic layer was then washed with brine (10 mL) and dried over anhydrous MgSO₄ and Na₂SO₄ for 5 minutes. Phase separation occurred. The supernatant was transferred to a separatory funnel and the lower aqueous layer was removed. The upper organic layer was dried again over anhydrous MgSO₄ and Na₂SO₄. Removal of the drying agent followed by evaporation of solvent gave the crude bromophthalane as a brown oil. This oil was redissolved in toluene (10 mL) and then concentrated on rotary evaporator. Subsequent drying under vacuum provided 1.25 g (96%) of the crude bromophthalane as a brown oil.

### EXAMPLE 8

### Preparation of Escitalopram

To a 10 mL rbf containing the bromophthalane produced in Example 7 (200 mg, 0.529 mmol) and CuCN (190 mg, 4 eq) was charged 2 mL of dimethylacetamide. The resultant mixture was heated to ˜150° C. under nitrogen with moderate stirring to a give a light greenish yellow solution. This solution was kept at ˜150° C. for 21 hours to produce a dark brown solution. After cooling to room temperature, this solution was partitioned between toluene (10 mL) and 50% conc. NH₄OH (10 mL) and stirred vigorously for 10 minutes. The lower dark blue aqueous layer was removed and the organic layer was washed again with another 10 mL of 50% conc. NH₄OH for 10 minutes. This washing operation was repeated one more time. The organic phase was then washed with brine (10 mL) and dried over anhydrous Na₂SO₄. Removal of the drying agent followed by evaporation of solvent gave crude Escitalopram as a brown oil (˜150 mg).

The crude Escitalopram was purified by column chromatography (silica gel 60, 70-230 mesh size, 3.5 g). The column was packed with 10% (10% NH₄OH in MeOH) in EtOAc. After the sample was loaded, the column was eluted, sequentially, with EtOAc (4×4.5 mL fractions), 10% (10% NH₄OH in MeOH) in EtOAc (12×2.5 mL fractions) and 15% (10% NH₄OH in MeOH) in EtOAc (6×2.5 mL fractions). Appropriate fractions were combined and the solvent was evaporated under reduced pressure to give a brown oil. Subsequent drying of this material under vacuum gave 113 mg of purified Escitalopram as a brown syrup. ¹H nmr analysis of this material showed that it contains about 15% of the phthalane starting material. Taking into account the amount of starting material present, the yield of purified Escitalopram was about 60%.

### EXAMPLE 9

### Preparation of Escitalopram Oxalate

Escitalopram (113 mg, containing about 15% bromophthalane) produced in Example 8 was mixed with oxalic acid dihydrate (44 mg, 0.349 mmol) in a 10 mL rbf. This mixture was dissolved in 2 mL of warm acetone to give a very pale brown solution. Removal of solvent under reduced pressure produced a white foam, which was triturated with EtOAc (˜2 mL) to generate a heavy white suspension. The solvent was evaporated and the resulting solid residue was triturated with 1 mL of acetone and 5 mL of EtOAc followed by stirring for 5 minutes. The white solid was collected by suction filtration and was dried under vacuum to afford 130 mg of the expected oxalic acid salt as an off-white powder. As expected, ¹H nmr analysis of this material showed that it contains about 15% of the salt derived from the bromo phthalane starting material of Example 8.

The specific rotation of the oxalic acid salt of Escitalopram produced in this example was found to be [α]_{D}+10.1° (at 20° C., c 0.95 in MeOH). The [α]_{D} reported in the literature for Escitalopram oxalate is +12.31° (c 1 in MeOH).

Although the invention has been described in connection with certain preferred embodiments, it is not limited thereto. Rather, the invention includes all embodiments which may fall within the scope of the following claims.

1. A process for preparing enantiomerically enriched (4-bromo-2-(hydroxymethyl)phenyl)-(4-fluorophenyl)methanol from racemic (4-bromo-2-(hydroxymethyl)phenyl)-(4-fluorophenyl)methanol, comprising:
(a) converting said racemic (4-bromo-2-(hydroxymethyl)phenyl)-(4-fluorophenyl)methanol to a racemic monoester intermediate by reaction with a carboxylic acid or a reactive derivative thereof;
(b) reacting said racemic monoester intermediate with an optically active acid to form a salt of said racemic monoester intermediate;
(c) crystallization of said salt to recover an enantiomerically enriched, crystalline form of said salt;
(d) neutralization of said salt to give an enantiomerically enriched form of said monoester intermediate; and
(e) hydrolysis of the enantiomerically enriched form of said monoester intermediate to produce said enantiomerically enriched (4-bromo-2-(hydroxymethyl)phenyl)-(4-fluorophenyl)methanol.

2. The process of claim 1, wherein the (4-bromo-2-(hydroxymethyl)phenyl)-(4-fluorophenyl)methanol produced in step (e) is enriched in an enantiomer which can be converted to escitalopram by dehydration and by substitution of bromine by a nitrile group.

3. The process of claim 1, wherein step (a) comprises reaction of said racemic (4-bromo-2-(hydroxymethyl)phenyl)-(4-fluorophenyl)methanol with a reactive derivative of a carboxylic acid, said reactive derivative being selected from the group comprising acid chlorides and acid anhydrides.

4. The process of claim 3, wherein said step (a) comprises reaction of said racemic (4-bromo-2-(hydroxymethyl)phenyl)-(4-fluorophenyl)methanol with acetic anhydride to form the monoacetate ester of said racemic (4-bromo-2-(hydroxymethyl)phenyl)-(4-fluorophenyl)methanol.

5. The process of claim 1, wherein said optically active acid is di-p-toluoyl tartaric acid.

6. The process of claim 5, wherein said optically active acid is (+)-di-p-toluoyl tartaric acid.

7. The monoacetate ester of (4-bromo-2-(hydroxymethyl)phenyl)-(4-fluorophenyl)methanol and salts thereof.

8. The ester of claim 7, being enantiomerically enriched.

9. The ester of claim 8, being enriched in an enantiomer which can be converted to escitalopram by dehydration and by substitution of bromine by a nitrile group.

10. The ester of claim 9, wherein said salt is the (+)-di-p-toluoyl tartaric acid salt of said monoacetate ester.

11. A process for preparing escitalopram, comprising:
(a) reacting 5-bromophthalide with 4-fluoro-phenylmagnesium bromide to produce 4-bromo-2-hydroxymethyl-4′-fluorobenzophenone;
(b) reacting said 4-bromo-2-hydroxymethyl-4′-fluorobenzophenone with 3-dimethylaminopropyl magnesium chloride to produce racemic (4-bromo-2-(hydroxymethyl)phenyl)-(4-fluorophenyl)methanol;
(c) converting said racemic (4-bromo-2-(hydroxymethyl)phenyl)-(4-fluorophenyl)methanol to a racemic monoester intermediate by reaction with a carboxylic acid or a reactive derivative thereof;
(d) reacting said racemic monoester intermediate with an optically active acid to form a salt of said racemic monoester intermediate;
(e) crystallization of said salt to recover an enantiomerically enriched, crystalline form of said salt;
(f) neutralization of said salt to give an enantiomerically enriched form of said monoester intermediate;
(g) hydrolysis of the enantiomerically enriched form of said monoester intermediate to produce enantiomerically enriched (4-bromo-2-(hydroxymethyl)phenyl)-(4-fluorophenyl)methanol;
(h) dehydration of said enantiomerically enriched (4-bromo-2-(hydroxymethyl)phenyl)-(4-fluorophenyl)methanol to produce enantiomerically enriched 1-(4′-fluorophenyl)-1-(3-dimethylaminopropyl)-5-bromophthalane; and
(i) replacement of bromine by a nitrile group to produce escitalopram.

12. The process of claim 11, wherein step (c) comprises reaction of said racemic (4-bromo-2-(hydroxymethyl)phenyl)-(4-fluorophenyl)methanol with a reactive derivative of a carboxylic acid, said reactive derivative being selected from the group comprising acid chlorides and acid anhydrides.

13. The process of claim 12, wherein said step (c) comprises reaction of said racemic (4-bromo-2-(hydroxymethyl)phenyl)-(4-fluorophenyl)methanol with acetic anhydride to form the monoacetate ester of said racemic (4-bromo-2-(hydroxymethyl)phenyl)-(4-fluorophenyl)methanol.

14. The process of claim 11, wherein said optically active acid is di-p-toluoyl tartaric acid.

15. The process of claim 14, wherein said optically active acid is (+)-di-p-toluoyl tartaric acid.
